(19) United States
(12) Patent Application Publication
(10) Pub. No.: US 2016/0227998 A1
(43) Pub. Date: Aug. 11, 2016

(54) ROTATING SLIT-SCAN FUNDUS IMAGER

(71) Applicant: Carl Zeiss Meditec, Inc., Dublin, CA (US)

(72) Inventors: Tilman SCHMOLL, Dublin, CA (US); Keith E. O'HARA, Pleasanton, CA (US)

(21) Appl. No.: 15/012,014

(22) Filed: Feb. 1, 2016

Related U.S. Application Data
(60) Provisional application No. 62/112,917, filed on Feb. 6, 2015.

(51) Int. Cl. A61B 3/12; A61B 3/00; A61B 3/15; A61B 3/14

(52) U.S. Cl. CPC A61B 3/12; A61B 3/14; A61B 3/0025; A61B 3/156

(57) ABSTRACT

An improved fundus imager for recording an image of a fundus of an eye is described. A line from a point on the fundus at a center of the image to a center of a pupil of the eye defines an axis. The fundus imager includes at least a source of illuminating light, optics for directing the illuminating light to the fundus of the eye, and a rotating element for rotating the illuminating about the axis such that the illuminating light illuminates the fundus at a plurality of locations around the axis. In a preferred embodiment of the present application, the rotating element is a rotating slit-shaped illumination aperture. In an alternate embodiment, the rotating element is a Dove prism for rotating the illuminating light on the fundus.

### PRIORITY

This application claims priority to U.S. Provisional Application Ser. No. 62/112,917, filed Feb. 6, 2015, the disclosure of which is incorporated herein by reference.

### TECHOLOGICAL FIELD

The present application concerns the design of fundus imagers.

### BACKGROUND

In a slit-scan ophthalmoscope, the illumination rays are kept separate from the lines-of-sight of the currently-exposed portion of the detector so that the image is free of haze from the anterior segment of the eye. This ophthalmoscope makes a stripe-shaped partition of the patient pupil, devoting some of the pupil to illuminating rays and some to detected rays. One such slit-scan ophthalmoscope is described by Humphrey, U.S. Pat. No. 4,732,466 (hereby incorporated by reference). U.S. Pat. No. 8,488,895 (also hereby incorporated by reference) describes further embodiments of a slit-scanning arrangement.

The slit-shaped illumination and viewing regions are scanned across the retina, for example from bottom to top as depicted in FIG. 1. In this figure, it should be noted that reference numeral 101 represents the retina, reference numeral 102 represents the cornea and crystalline lens, illumination rays are represented by reference numeral 104a and 104b (individually or collectively referred to herein as 104), and the detected rays are represented by reference numeral 103. It should further be noted that throughout this disclosure and in all figures, reference numeral 104 will be used to refer to the illumination rays, and reference numeral 103 will be used to refer to the returned, reflected, or detected rays. Such a scanning procedure builds an image of the entire retina. The apertures creating this separation of optical paths are relay-imaged to the patient pupil. With practical optical designs, this imaging can have deficiencies. The same lenses (e.g., the two lenses in front of eye 351 in FIG. 3b) that relay the aperture to the pupil also transport the retinal image light, and the goal of good retinal image quality competes somewhat with quality of relaying the aperture to the pupil.

The above discussed scanning procedure works when the illuminated strip is vertically in the center of the field-of-view where the illuminating rays 104 and detected rays 103 are well separated at the patient pupil (see FIG. 2a). However, when the illumination is scanned to the upper or lower retina, aberrations in the lenses can easily cause the illumination rays to overlap the detection paths (see for example, FIGS. 2b and 2c).

The beam footprints in FIG. 2 show results of computed ray-tracing from the illuminating line and row of detector pixels, through their respective apertures to the patient pupil, with a 6-mm diameter circle displayed for reference. The footprint in 1) FIG. 2a is in the patient pupil for a centered fan of rays, 2) FIG. 2b is for beams deflected to illuminate and view the lower retina, and 3) FIG. 2c is at the middle of the eye lens, when the beams are deflected toward the lower retina. As can be seen from footprints in FIGS. 2b and 2c, the viewing paths are not clear of the illumination paths, due to which illumination light scattered from the eye lens appears as haze in the resulting images.

### SUMMARY

According to one aspect of the subject matter described in the present application, an improved fundus imaging apparatus is discussed that rotates the pattern of illuminating and detected rays about the optical axis of the imaging apparatus, rather than scan as usual with beams pivoting about an axis in the plane of the patient pupil. Optical aberrations in the relay of the aperture to the pupil cause radial errors in the locations of the light rays, when using lenses with rotational symmetry. Radial errors in the positions of the rays at the pupil do not reduce the separation between illumination and detection, to the extent that the boundary between illumination and detection paths lies near a radial line through the optical axis. Rotation of the paths about the optical axis allows the boundary to remain near radial, keeping better separation between illumination and detection.

In the fundus imaging apparatus discussed herein, the slit apertures on illumination and detection paths are optically conjugate to (i.e. optically imaged onto) the retina, so that the edges of the slit are sharp in the image. Further, a third rotating element (an obscuration element for blocking the illuminating light from overlapping in an anterior segment of the eye with light scattered by the fundus) is added. The obscuration element is synchronously rotated with the illumination aperture about the optical axis.

In a preferred embodiment, the slit illumination that is imaged to the retina is narrower at the center than at its ends, which are wider. This increases the uniformity of integrated exposure.

### BRIEF DESCRIPTION OF THE DRAWINGS

FIG. 1 is an exemplary prior-art schematic of the interaction between illumination and detected rays, and the optics of an eye comprising cornea, lens, pupil, and the retina.

FIGS. 2a-2c show three beam footprints, each of which illustrating the separation between illumination rays and detected rays in the pupil according to an exemplary prior-art fundus imaging apparatus. The separation depends upon where the retina is illuminated. The diameter of the circle in each of the three cases is 6 mm. The footprint in FIG. 2a is in the patient pupil for a centered fan of rays; FIG. 2b is for beams deflected to illuminate and view the lower retina; FIG. 2c is at the middle of the eye lens in depth, when the beams are deflected toward the lower retina.

FIG. 3a is a system schematic showing the various components that comprise a fundus imager according to one aspect of the present invention. FIG. 3b is one of the possible optical configurations of the fundus imager discussed herein.

FIG. 4 is an expanded view of part of the optical configuration of the fundus imager shown in FIG. 3b. In particular, FIG. 4 depicts a rectangular obscuration element that is placed into the illumination beam to reduce overlap at the pupil of the eye between detected rays and illumination rays.

FIGS. 5a-5c show three beam footprints of illumination and detected rays that are achieved using the fundus imager discussed herein. In particular, FIG. 5a shows the beam footprint at the mirror (indicated by reference numeral 301 in FIG. 3a or 4); FIG. 5b shows the beam footprint at the patient pupil; and FIG. 5c shows the beam footprint at the posterior side of the eye lens.

FIG. 6 is an expanded view of part of the optical configuration of the fundus imager shown in FIG. 3b. In particular, FIG. 6 shows pattern of the illumination and detected rays relative to the eye of a patient.

FIG. 7 shows an exemplary rotating slit according to one aspect of the present invention.

FIG. 8 is an expanded view of part of the optical configuration of the fundus imager shown in FIG. 3b. In particular, FIG. 8 shows an ophthalmic lens, an eye of a patient, and pattern of the illumination and detected rays relative to the lens and the eye.

FIGS. 9a and 9b show two exemplary placements of a Dove prism in the fundus imager configuration of FIG. 3b. In particular, FIG. 9a shows a Dove prism placed in the illumination path of the fundus imager while FIG. 9b shows the Dove prism placed in the illumination as well as detection paths of the fundus imager.

### DETAILED DESCRIPTION

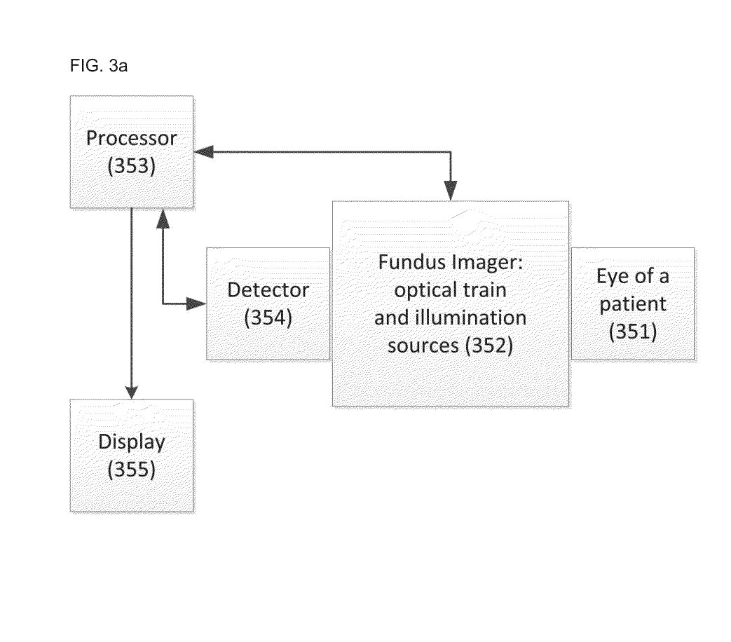

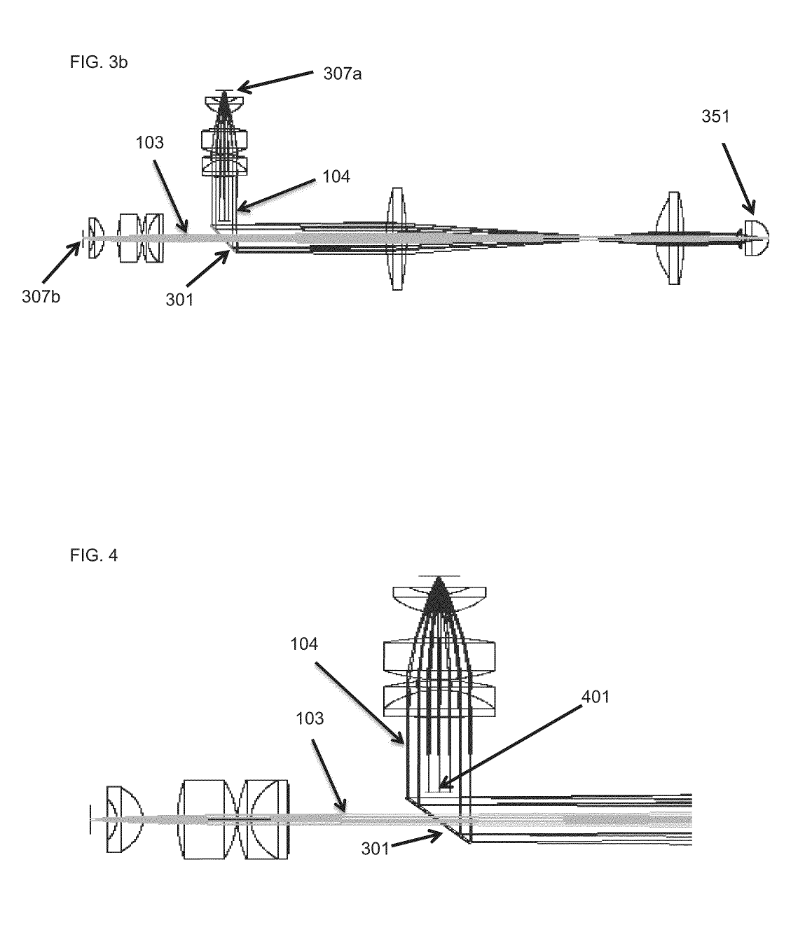

An exemplary fundus imager or imaging system is schematically displayed in FIG. 3a. As depicted, it includes at least: an optical train 352, which includes one or more sources of illumination; a detector 354; and a processor 353 that controls the system as well as the subsequent data and to which a display 355 is connected. An eye 351 of a patient is the object to be imaged by the fundus imaging system.

FIG. 3b shows an example possible optical layout of the fundus imaging system. Here, illumination rays are indicated by reference numeral 104 (shown in black), detected rays indicated by reference numeral 103 (shown in gray), and the eye 351 at the extreme right of the schematic. The illumination and detection paths are in this embodiment divided using a mirror 301 with a hole in the center. Alternatively, a beamsplitter plate can be used, in some instances, to separate illumination and detection light.

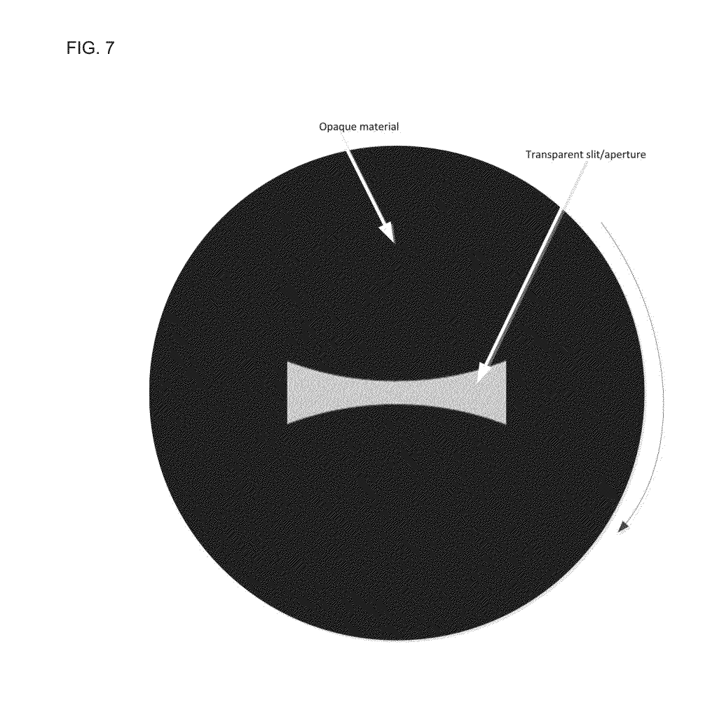

Each of the illumination and detection paths has a rotating element, such as a slit-shaped rotating aperture (for example, see FIG. 7). For instance, with reference to FIG. 3b, an illumination aperture is indicated by reference numeral 307a in the illumination path 104 and a detection aperture is indicated by reference numeral 307b in the detection path 103. The illumination aperture at 307a defines the shape of the illumination rays when it reaches the retina. The detection aperture at 307b defines the shape of the rays reflected and/or scattered back from the retina (referred to herein as detection rays) to include nominally the illuminated portion of retina only, by blocking light that comes from other locations on the retina from reaching the detector array. A rectangular obscuration element 401 (see FIG. 4) is placed near the dividing mirror 301that can serve as a pupil-splitting aperture for blocking illumination rays that would cross the detection path in the anterior segment of the eye. The rectangular obscuration element 401 has its longer dimension parallel to the illumination aperture 307a, and rotates in synchrony with the apertures 307a and 307b. This rotating obscuration element 401 defines the shape of the illumination paths at the plane of the patient pupil. By rotating this obscuration element 401 in synchrony with the slit-shaped illumination and detection apertures 307a and 307b, the entire three-dimensional pattern of illumination and detection beam paths is rotated in synchrony, which keeps them clear of each other through the full depth of the anterior segment of the eye 351.

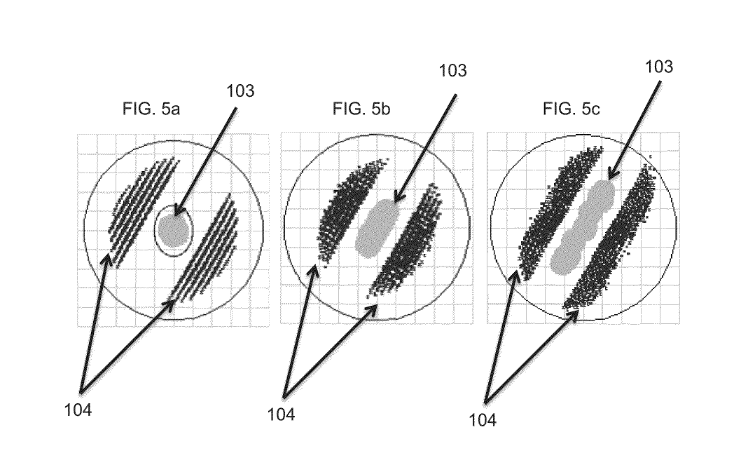

The footprints in FIGS. 5a-5c show the arrangement of the illumination and detection beams at various locations along the optical train depicted in FIGS. 3b and 4. Specifically, FIG. 5a represents the footprint at the diving mirror 301; FIG. 5b represents the footprint at the patient pupil; and FIG. 5c represents the footprint at the posterior side of the eye lens. A 6-mm diameter reference circle is also given on each of the footprints. The separate gray circles (representing the detection rays 103) approximately centered in each of FIGS. 5a-c correspond to the discrete points on the retina from which viewing paths are traced. This set of imaged points falls within a slit-aperture with 50:1 aspect ratio. As seen, viewing paths remain clear of the illumination rays through the full depth of the anterior segment of the eye.

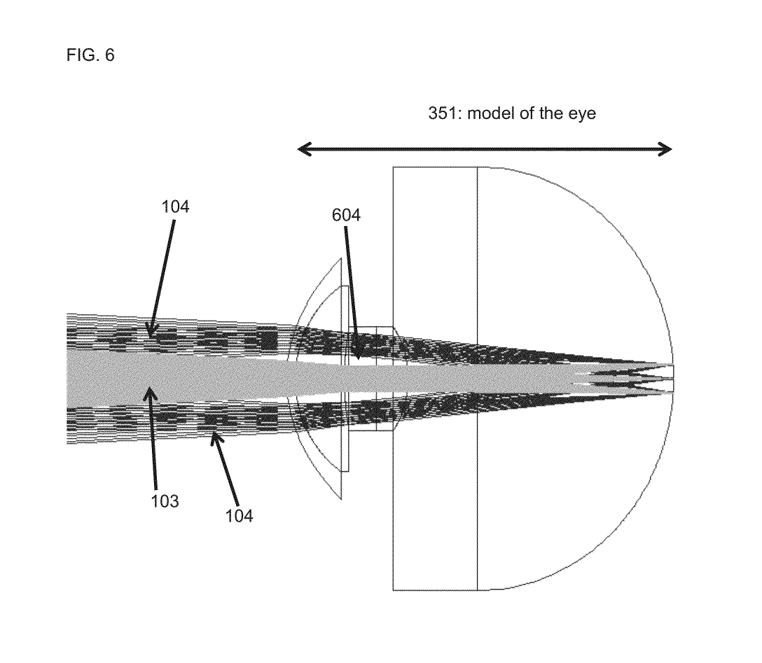

FIG. 6 shows an enlargement of the eye portion 351 of the schematic of FIG. 3b. This depiction is of a model of the eye 351 incorporating representations for the cornea, location of the pupil, the crystalline lens, and the retina (with a typical retinal curvature). The illumination beams are indicated by 104; the detected beams by 103; and the desired gap or region of non-overlap between these two beams at the location of the pupil by 604.

In a preferred embodiment, the slit-shaped illumination aperture 307a is sufficiently narrow that it restricts the range of ray angles to within ±3° of the central illumination plane. This restriction on ray angles limits the distance that the illumination rays impinge into the nominally-clear zone for detection to about 0.25 mm, through the depth of the anterior segment of the human eye. Similarly, in a preferred embodiment, the slit-shaped detection aperture 307b is sufficiently narrow to restrict the viewing paths to within ±3° of the illumination plane. Then the viewing paths slope only about 0.25 mm toward the illumination rays, through the depth of the anterior segment. In this way, a gap (for example, the gap 604 in FIG. 6) of about 0.5 mm between illumination and detection patterns at the pupil suffices to keep the paths clear through the scattering structures of the cornea and lens in the human anterior segment. Moreover, such a gap can be allowed while still fitting illumination and detection within the area of the human pupil.

In some embodiments, each of the illumination aperture 307a and the detection aperture 307b rotates at a speed of 100 Hz or faster, so that at least one half-cycle of the apertures cover and detect the entire field-of-view on the retina within 5 ms. This speed allows a frame exposure to complete before motion of the human eye causes noticeable motion blur. The exposure can be repeated, for example using separate red, green, and blue illumination flashes (or any other wavelength of light, e.g., infrared) to build a composite color image, for up to 0.25 seconds before a typical blink reflex. Each exposure in the set is free of motion blur and the captured frames can be registered to each other before combining to build an image free of motion blur.

Front-Lens Reflections:

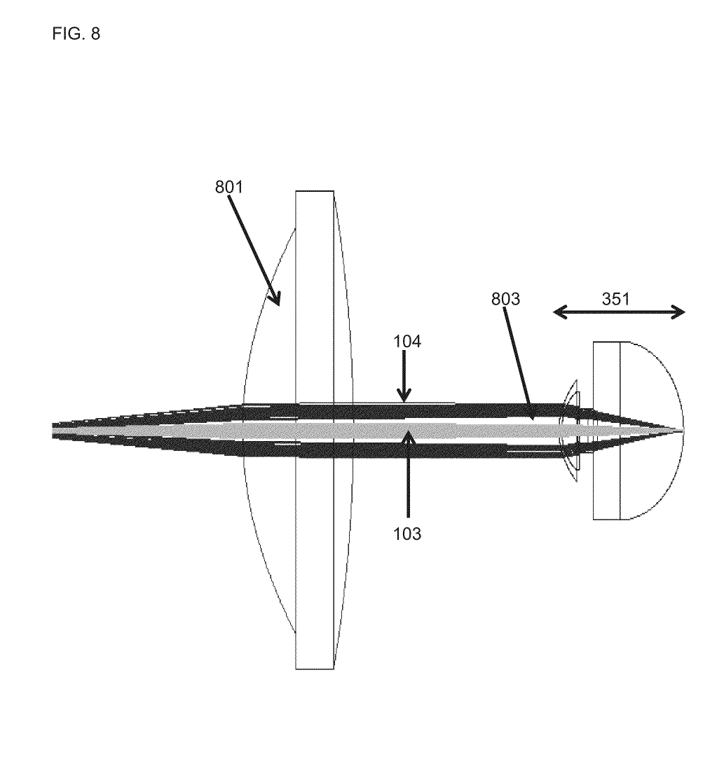

Some illuminating light reflects from the surfaces of an ophthalmic lens (e.g., ophthalmic lens 801 in FIG. 8) placed just in front of the patient's eye 351. It is desirable to have considerable space between the patient's eye and this lens so that even with a reasonably thin fan of rays, within ±3° of the central illumination plane, the illumination rays have impinged on the detection paths at the distance of the ophthalmic lens from the patient pupil.

Due to the curvature of the lenses, however, only their central portions tend to reflect light in the direction of the fundus imager's collection or detection aperture. If only the central portion of the illumination aperture (e.g., rotating slit-shaped illumination aperture depicted in FIG. 7), is narrowed sufficiently to keep illumination and detection paths separate through the instrument lenses, as well as through the anterior segment of the eye, then reflections from the instrument are eliminated as well as scattering from the eye.

FIG. 8 is an enlargement of the optical layout depicted in FIG. 3b illustrating the illumination and detection beam patterns relative to the ophthalmic lens 801 and the optical representation for the eye 351. A desired gap between the illumination beam 104 and the detected beam 103 is indicated by reference numeral 803.

For a single ophthalmic lens on the common path between illumination and detection, at a distance 40 mm from the patient pupil, the 0.5-mm gap between illumination and detection at the patient pupil can be maintained as a finite gap though the ophthalmic lens if the central slit is narrowed to reduce the ray spread from the usual 6° down to 0.5/40=0.0125 radians=0.7°.

If the instrument is also correcting refractive error of the patient, it will focus the illumination paths so that they converge or diverge as needed to be in focus at the patient's retina. That convergence or divergence will change the angle of the illumination paths relative to the detection paths. Instead of parallel ray fans outside the patient's eye, when refraction error is corrected the ray fans cross at the patient's near-point. Correction for refractive error reduces the length over which the illumination and detection paths remain separate, as these paths overlap completely at the patient's near point. The angle of illumination can be chosen, by choosing the central width of the slit, to keep the paths separate over any desired portion of the distance from the patient pupil to his near-point.

Alternate Fundus Imaging Design:

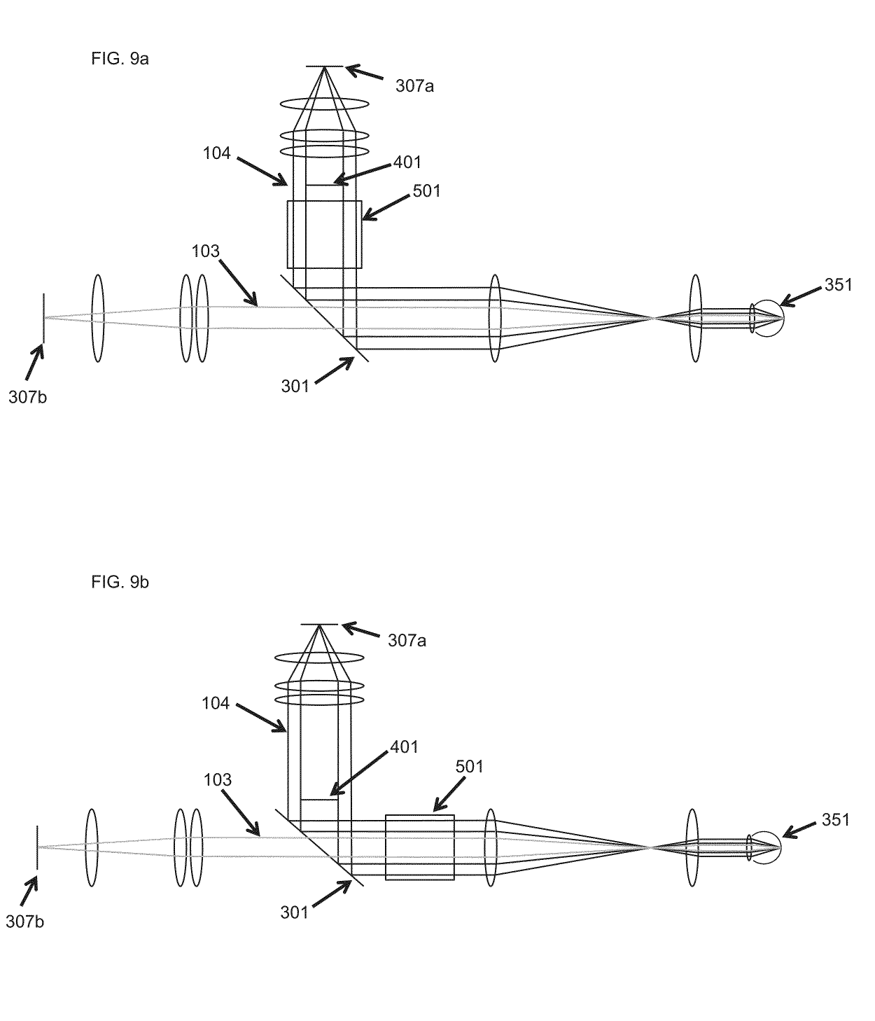

In an alternate embodiment of the present invention, rather than rotating illumination or detection apertures, their ray paths can be rotated. A Dove prism, for example, rotates the field of view seen through the prism, at twice the rate of rotation of the Dove prism itself. In some embodiments, the Dove prism can be placed in the illumination arm, i.e. in front of the optical element that separates illumination and detection light. For example, with reference to FIG. 9a, a Dove prism 501 can be placed in the illumination path 104 for rotating the beam pattern resulting from the illumination aperture 307a and the obscuration element 401. One may use an optional detection aperture in front of the detector, which rotates in synchrony with the Dove prism. In some embodiments, a Dove prism can be placed in the portion where detection and illumination rays are joined (for example, see FIG. 9b) to rotate the entire beam pattern. Since the detection beam pattern is rotated along with the illumination beam pattern, the image of the retina on the detector array also rotates such that the area of illumination remains stationary on the detector array. In contrast to the embodiments with the rotating apertures or Dove prism placed solely in the illumination arm, one would in this case have to acquire many short frame acquisitions per 180° rotation of the slit. An image is built from several exposures by the detector array, each exposure contributing a radial-stripe portion of the image. The illumination should be strobed, or the rotation of the prism stepped, to limit motion-blur in the periphery of the image. Strobed illumination and stepped-rotation may be unnecessary with an area detector that is able to acquire at very high frame rates.

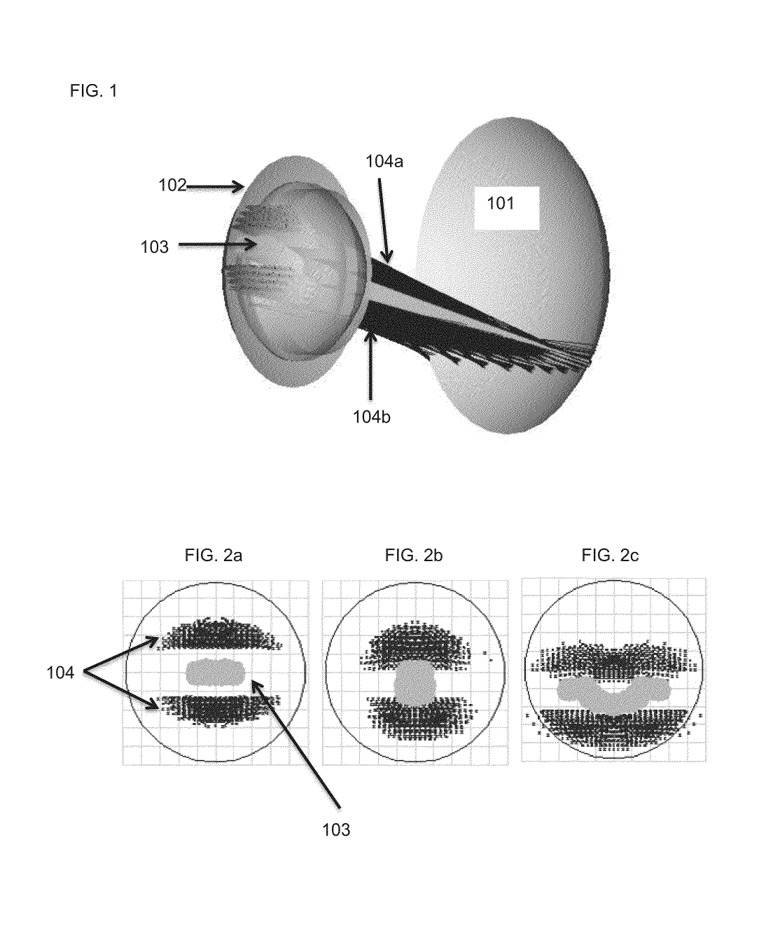

In another embodiment, only one can choose to use only one portion of the pupil for illumination instead of two (e.g., using only path 104a in FIG. 1) so that less pupil-area is wasted for gaps between illumination and detection. This design may fit best in an implementation where the ray patterns are rotated optically (i.e., where the illumination and detection paths have been joined) to save the complexity of offsetting the hole in the hole-mirror (e.g., the mirror 301), and rotating that mirror in synchrony with the other apertures. With this asymmetric splitting of the pupil, it is helpful to offset the slits that are conjugate to the image planes slightly toward the viewing side of the pupil. When the illumination beam enters the superior pupil, for example, it illuminates a region of retina up to one-half slit-width toward the inferior. In this way, the reflection from the flatter patient-side surface of the ophthalmic lens would appear to come from the superior retina, and be blocked by the slit in the imaging path which is shifted to accept light from the inferior retina.

In the above description, for purposes of explanation, numerous specific details are set forth in order to provide a thorough understanding of the specification. It should be apparent, however, that the subject matter of the present application can be practiced without these specific details. It should be understood that the reference in the specification to “one embodiment”, “some embodiments”, or “an embodiment” means that a particular feature, structure, or characteristic described in connection with the embodiment is included in one or more embodiments of the description. The appearances of the phrase “in one embodiment” or “in some embodiments” in various places in the specification are not necessarily all referring to the same embodiment(s).

The foregoing description of the embodiments of the present subject matter has been presented for the purposes of illustration and description. It is not intended to be exhaustive or to limit the present embodiment of subject matter to the precise form disclosed. Many modifications and variations are possible in light of the above teaching. It is intended that the scope of the present embodiment of subject matter be limited not by this detailed description, but rather by the claims of this application. As will be understood by those familiar with the art, the present subject matter may be embodied in other specific forms without departing from the spirit or essential characteristics thereof.

The following references are hereby incorporated by reference:

U.S. Pat. No. 4,135,791

U.S. Pat. No. 4,732,466

U.S. Pat. No. 8,488,895

What is claimed is:

1. A fundus imager for recording an image of a fundus of an eye, the image having a center and the eye having a pupil, wherein the line from the point on the fundus at the center of the image to the center of the pupil defines an axis, said fundus imager comprising:
a source of illuminating light;
optics for directing the illuminating light to the fundus of the eye;
a rotating element for rotating the illuminating light about said axis, so that the illuminating light illuminates the fundus at a plurality of locations around the axis;
return optics for directing light scattered from the fundus of the eye to a detector, said detector generating signals in response thereto; and
a processor for processing the signals and for generating the image of the fundus of the eye based on the processed signals.

2. A fundus imager as recited in claim 1 further comprising:
a pupil splitting aperture for separating the illuminating light and the light scattered from the fundus.

3. A fundus imager as recited in claim 1, in which the fundus is illuminated with a stripe of illumination and rotating the illuminating light about said axis illuminates a disk shaped area on the fundus of the eye.

4. A fundus imager as recited in claim 1, wherein the optics for directing the illuminating light includes a slit-shaped illumination aperture that illuminates a stripe on the fundus of the eye.

5. A fundus imager as recited in claim 4, wherein the slit-shaped illumination aperture is optically conjugate to the retina of the eye.

6. A fundus imager as recited in claim 4, wherein the slit-shaped illumination aperture has a central portion and a peripheral portion, said central portion has a width less than half the width of the peripheral portion.

7. A fundus imager as recited in claim 4, wherein the slit-shaped illumination aperture is sufficiently narrow to restrict the range of ray angles to within ±3° of the central illumination plane.

8. A fundus imager as recited in claim 1, wherein the rotating element is a rotating slit-shaped illumination aperture.

9. A fundus imager as recited in claim 8, wherein the rotating slit-shaped illumination aperture is optically conjugate to the retina of the eye.

10. A fundus imager as recited in claims 8, wherein the rotating slit-shaped illumination aperture has a central portion and a peripheral portion, said central portion has a width less than half the width of the peripheral portion.

11. A fundus imager as recited in claim 8, wherein the rotating slit-shaped illumination aperture is sufficiently narrow to restrict the range of ray angles to within ±3° of the central illumination plane.

12. A fundus imager as recited in claim 1 further comprising:
an obscuration element for blocking the illuminating light from overlapping in an anterior segment of the eye with the light scattered from the fundus.

13. A fundus imager as recited in claim 12, wherein the obscuration element is synchronously rotated with the rotating element.

14. A fundus imager as recited in claims 1, wherein the rotating element is a Dove prism for rotating the illuminating light on the fundus.

15. A fundus imager as recited in claims 1 further comprising:
a detection aperture for blocking stray light, said aperture is synchronously rotated with the rotating element.

16. A fundus imager as recited in claim 15, wherein the detection aperture has a central portion and a peripheral portion, said central portion has a width less than half the width of the peripheral portion.

17. A fundus imager as recited in claim 15, wherein the detection aperture is optically conjugate to the retina of the eye.

18. A fundus imager as recited in claim 15, wherein the detection aperture is sufficiently narrow to restrict the viewing paths to within ±3° of the illumination plane.

19. A fundus imager as recited in claim 1, wherein the illuminating light is rotated at a speed of at least 100 Hz.
